(19) United States
(12) Patent Application Publication
(10) Pub. No.: US 2026/0053146 A1
(43) Pub. Date: Feb. 26, 2026

(54) MASTOPARAN PEPTIDES AS TREATMENT AGAINST LIBERIBACTER-CAUSED CROP DISEASES

(71) Applicant: The United States of America, as represented by the Secretary of Agriculture, Washington, DC (US)

(72) Inventors: MICHELLE L HECK, ITHACA, NY (US); LUKE A THOMPSON, ITHACA, NY (US); SAMUEL CORADETTI, ITHACA, NY (US)

(21) Appl. No.: 19/235,666

(22) Filed: Jun. 12, 2025

Related U.S. Application Data
(60) Provisional application No. 63/662,543, filed on Jun. 21, 2024.

(51) Int. Cl. A01N 63/50; A01P 1/00

(52) U.S. Cl. CPC A01N 63/50; A01P 1/00

(57) ABSTRACT

The disclosure relates to bioactive compositions comprising at least one mastoparan peptide and a carrier and/or adjuvant, or at least one modified mastoparan peptide and use of such bioactive compositions to protect plants from infection by pests or pathogens. More specifically, the disclosure provides a method for treating and/or ameliorating a pest and/or pathogen in a Citrus or Solanaceae plant or plant part in need thereof, the method comprising treating the plant or plant part with an effective amount of a bioactive composition comprising at least one mastoparan peptide and a carrier and/or adjuvant, or at least one modified mastoparan peptide to treat and/or ameliorate the pest and/or pathogen in the Citrus or Solanaceae plant.

### CROSS-REFERENCE TO RELATED APPLICATIONS

This application claims priority benefit from U.S. Provisional Application No. 63/662,543, filed Jun. 21, 2024. The contents of this patent application are hereby expressly incorporated by reference in their entirety.

### FIELD

The disclosure relates to the field of agricultural biotechnology, in particular to bioactive compositions comprising at least one modified mastoparan peptide or a mastoparan peptide and an adjuvant or carrier, and methods for using such bioactive compositions to prevent, treat, and/or ameliorate plant pests and pathogens.

### SEQUENCE LISTING

The instant application contains a Sequence Listing XML required by 37 C.F.R. § 1.831(a) which has been submitted in XML file format via the USPTO patent electronic filing system, and is hereby incorporated by reference in its entirety. The XML file was created on Jun. 21, 2024, is named 0078_23_Sequence_Listing, and has 36,426 bytes.

### BACKGROUND

Mastoparans are a family of natural antimicrobial peptides identified in wasp venom. Mastoparans have been of medical interest for several decades due to their immuno-stimulatory effects on mast cells. Some of them have more recently been recognized as having antimicrobial activities as well, with multiple reports of activity against diverse bacterial and fungal species. There is no indication that any of the mastoparan peptides are effective in preventing, treating, and/or ameliorating plant pests and pathogens.

Liberibacter species include several phloem-limited, insect borne-plant pathogens that are the causative agents of several economically-destructive crop diseases including Citrus Greening Disease and Zebra Chip Disease of potato.

There are no established, cost-effective treatments to treat or prevent Liberibacter infections. Thus far, the most successful mitigation efforts for Citrus Greening Disease consist of nutritional support for infected trees, which slows disease progression but does not prevent the decline and death of all infected trees. Experimental treatment by direct injection of oxytetracycline has been reported to improve tree health, but Liberibacter persists in infected trees and the long-term success of this treatment has not been established. Without any effective treatment, Citrus Greening has devastated the Florida citrus industry with estimated losses exceeding $1 billion annually (doi.org/10.21273/HORTSCI14696-19). Losses due to Zebra Chip Disease (for which the only viable control strategies consist of aggressive insect management) are less well quantified, though it has become a common problem throughout the western United States, a multibillion dollar market.

Thus any effective new treatment against Liberibacter in the field will have the potential to restore billions of dollars of agricultural activity annually. Therefore, new methods of controlling Liberibacter are urgently needed.

### SUMMARY

Provided herein are bioactive compositions comprising at least one mastoparan peptide and an adjuvant or carrier, or comprising at least one modified mastoparan peptide, and methods of protecting plants from pest and/or pathogen infection using such.

In an embodiment, the disclosure relates to a bioactive composition comprising at least one modified mastoparan peptide. In some embodiments of the disclosure, the at least one modified mastoparan peptide in the bioactive composition is modified by an N-terminal modification, an N-terminal substitution, a C-terminal modification, a C-terminal substitution, the presence of a D-amino acid, the presence of an unnatural amino acid, cyclization, or a mixture thereof. In some embodiments of the disclosure, the at least one modified mastoparan peptide has the amino acid sequence as set forth in any one of SEQ ID NO: 1 to SEQ ID NO: 41, or a mixture thereof.

In an embodiment, the disclosure relates to a bioactive composition comprising at least one mastoparan peptide and an adjuvant and/or carrier. In some embodiments of the disclosure, the at least one mastoparan peptide in the composition comprising a masotoparan peptide and an adjuvant and/or carrier has the amino acid sequence as set forth in any one of SEQ ID NO: 1 to SEQ ID NO: 41, or a mixture thereof.

In an embodiment, the disclosure relates to a method for treating and/or ameliorating a pest and/or pathogen in a subject in need thereof, the method comprising treating the subject with an effective amount of a bioactive composition comprising at least one modified mastoparan peptide, or a bioactive composition comprising at least one mastoparan peptide and an adjuvant and/or carrier to treat or ameliorate the pest and/or pathogen in the subject.

### BRIEF DESCRIPTION OF THE DRAWINGS

FIG. 1 depicts a graph of the L. crescens growth in media treated with 200 g/mL of the indicated compound. The X axis shows the relative grown, and the Y axis shows the compounds tested. Control (buffer solution with no peptides added); Mastoparan-R2 (set forth in SEQ ID NO: 1); Mastoparan-T (set forth in SEQ ID NO: 6); Mastoparan-AF (set forth in SEQ ID NO: 5); Mastoparan-M (set forth in SEQ ID NO: 11); Mastoparan-VT6 (set forth in SEQ ID NO: 4); Mastoparan-12d (set forth in SEQ ID NO: 7); Mastoparan-VT4 (set forth in SEQ ID NO: 9); Mastoparan-VT3 (set forth in SEQ ID NO: 12); Mastoparan-L (set forth in SEQ ID NO: 10); Mastoparan-VT2 (set forth in SEQ ID NO: 8); EMP-AF (set forth in SEQ ID NO: 14); MP-VB1 (set forth in SEQ ID NO: 13); Protopolybia-MPII (set forth in SEQ ID NO: 18); Protonectarina-MP (set forth in SEQ ID NO: 17); Mastoparan-X (set forth in SEQ ID NO: 21); Mastoparan-like_12b (set forth in SEQ ID NO: 23); PMM (set forth in SEQ ID NO: 19); PDD-A (set forth in SEQ ID NO: 16); Mastoparan-V1 (set forth in SEQ ID NO: 20); Mastoparan-V2 (set forth in SEQ ID NO: 15); Parapolybia-MP (set forth in SEQ ID NO: 22); Protopolybia-MPIII (set forth in SEQ ID NO: 24); PDD-B (set forth in SEQ ID NO: 29); MP (set forth in SEQ ID NO: 24); Protopolybia MPI (set forth in SEQ ID NO: 31); Polybia-MPII (set forth in SEQ ID NO: 28); Polybia-MPIII (set forth in SEQ ID NO: 26); Mastoparan-R3 (set forth in SEQ ID NO: 27); Polybia-MPI (set forth in SEQ ID NO: 33); EMP-ER (set forth in SEQ ID NO: 34); EMP-OD (set forth in SEQ ID NO: 36); Mastoparan-B (set forth in SEQ ID NO: 39); EMP-EM1 (set forth in SEQ ID NO: 38); Mastoparan-J (set forth in SEQ ID NO: 41); Mastoparan-VT5 (set forth in SEQ ID NO: 40).

FIG. 2 depicts a schematic representation of the detached leaf assay.

FIG. 3 depicts a graph of the CLas abundances from citron leaves treated with either a positive (polymyxin B, PMB), a negative control (buffer alone), or a mastoparan peptide. Very low titer samples (with about 1 to 10 copies per sample or ‘ND’, below detection level) are plotted separately for clarity. The Y axis presents the number of CLas 16S DNA copies. The X Axis presents the treatments: Buffer alone, EMP-AF; VT4; VT6; PMB (polymyxin B); EPM-ER; Protopolybia MPI; or Protopolybia MPI. The sequence of EMP-AF is set forth in SEQ ID NO: 14; the sequence of mastoparan VT4 is set forth in SEQ ID NO: 9; the sequence of mastoparan VT-6 is set forth in SEQ ID NO: 4; the sequence of EMP-ER is set forth in SEQ ID NO: 34; the sequence of Protopolybia MPI is set forth in SEQ ID NO:31; and the sequence of Protopolybia MPIII is set forth in SEQ ID NO: 18.

FIG. 4 depicts a graph of the CLas abundance in adult psyllids raised on CLas-infected citron leaves treated with buffer alone, PMB, or a mastoparan peptide. Very low titer samples (less than 1 copy per sample, ‘ND’) were plotted separately for clarity. The Y axis presents the number of CLas 16S DNA copies. The X Axis presents the treatments: Buffer alone, EMP-AF; VT4; VT6; PMB (polymyxin B); EPM-ER; Protopolybia MPI; or Protopolybia MPII.

### BRIEF DESCRIPTION OF THE SEQUENCE LISTING

The sequence identifier (SEQ ID NO), name, and amino acid sequence of the mastoparan disclosed herein are listed in Table 1 below.

TABLE 1

| SEQ | | |
|---|---|---|
| ID NO: | Name | Sequence |
| 1 | Mastoparan-R2 | LNFKALAALAKKIL |
| 2 | Mastoparan-C | LNLKALLAVAKKIL |
| 3 | Mastoparan-VT7 | INLKAIAALARNY |
| 4 | Mastoparan-VT6 | INLKAIAALVKKLL |
| 5 | Mastoparan-AF | INLKAIAALAKKLF |
| 6 | Mastoparan-T | INLKAIAAFAKKLL |
| 7 | Mastoparan-like_12d | INLKAIAAMAKKLL |
| 8 | Mastoparan-VT2 | NLKAIAALAKKLL |
| 9 | Mastoparan-VT4 | INLKAIAPLAKKLL |
| 10 | Mastoparan-L | INLKALAALAKKIL |
| 11 | Mastoparan-M | INLKAIAALAKKLL |
| 12 | Mastoparan-VT3 | INLKAITALAKKLL |
| 13 | MP-VB1t | INMKASAAVAKKLL |
| 14 | EMP-AF | INLLKIAKGIIKSL |
| 15 | Mastoparan-V2 | INWKKIKSLIKAAMS |
| 16 | PDD-A | INWKKIFEKVKNLV |
| 17 | Protonectarina-MP | INWKALLDAAKKVL |
| 18 | Protopolybia-MPII | INWKAIIEAAKQAL |
| 19 | PMM | INWKKIASIGKEVLKAL |
| 20 | Mastoparan-V1 | INWKKIKSIIKAAMN |
| 21 | Mastoparan-X | INWKGIAAMAKKLL |
| 22 | Parapolybia-MP | INWKKMAATALKMI |
| 23 | Mastoparan-like_12b | INWKGIAAMKKLL |
| 24 | Protopolybia-MPIII | INWLKLGKAVIDAL |
| 25 | Mastoparan-R1 | INWLKLGKKILGAI |
| 26 | Polybia-MPIII | INWLKLGKMVMDAL |
| 27 | Mastoparan-R3 | INWLKLGKQILGAL |
| 28 | Polybia-MPII | INWLKLGKMVIDAL |
| 29 | PDD-B | INWLKLGKKILGAL |
| 30 | MP | INWLKLGKKMMSAL |
| 31 | Protopolybia-MPI | INWLKLGKKVSAIL |
| 32 | Venom_protein_13b | IDWLKLGKMVMDVL |
| 33 | Polybia-MPI | IDWKKLLDAAKQIL |
| 34 | EMP-ER | FDIMGLIKKVAGAL |
| 35 | EMP-EF | FDVMGIIKKIAGAL |
| 36 | EMP-OD | GRILSFIKGLAEHL |
| 37 | EMP-EM2 | LKLLGIVKKVLGAI |
| 38 | EMP-EM1 | LKLMGIVKKVLGAL |
| 39 | Mastoparan-B | LKLKSIVSWAKKVL |
| 40 | Mastoparan-VT5 | VIVKAIATLSKKLL |
| 41 | Mastoparan-J | VDWKKIGQHILSVL |

### DETAILED DESCRIPTION

The present disclosure relates to bioactive compositions comprising at least one modified mastoparan peptide or comprising a mastoparan peptide and an adjuvant or carrier, and methods of using such bioactive compositions for treating and or ameliorating a pest and/or pathogen in a subject in need thereof.

The presently disclosed subject matter may be understood more readily by reference to the following detailed description taken in connection with the accompanying examples, which form a part of this disclosure. As employed above and throughout the disclosure, the following terms and abbreviations, unless otherwise indicated, shall be understood to have the following meanings.

Unless otherwise explained, all technical and scientific terms used herein have the same meaning as commonly understood by one of ordinary skill in the art to which this disclosure belongs. The singular terms “a”, “an”, and “the” include plural referents unless context clearly indicates otherwise. Similarly, the word “or” is intended to include “and” unless the context clearly indicate otherwise.

Unless otherwise indicated, all numbers expressing quantities of ingredients, properties such as molecular weight, reaction conditions, and so forth as used herein are to be understood as being modified in all instances by the term “about.” Accordingly, unless otherwise indicated, the numerical properties set forth herein are approximations that may vary depending on the desired properties sought to be obtained. Notwithstanding that the numerical ranges and parameters setting forth the broad Scope of the disclosure are approximations, the numerical values set forth in the specific examples are reported as precisely as possible. Any numerical values, however, inherently contain certain errors necessarily resulting from error found in their respective measurement.

As used herein, the term “about” is defined as plus or minus ten percent of a recited value. For example, about 1.0 g means 0.9 g to 1.1 g.

As used herein, the term “unnatural amino acid” or “UAA” refers to an amino acid not found in natural polypeptide chains. An UAA may be formed as a secondary metabolite in bacteria, fungi, plants, or marine organisms, or may be synthesized chemically. UAAs can be divided into two major groups: UAAs that are structurally similar to canonical amino acids and therefore frequently named as analogues; and surrogate UAAs that differ dramatically from canonical amino acids. Whatever group they belong to, their distinct chemical and biological characteristics that stem from their unusual side groups and/or D-stereochemistry make them highly attractive as biologically active molecules or building blocks.

As used herein, “bioactive” refers to a product derived from natural sources that may be used to control pests, pathogens, and/or weeds. The bioactive compositions disclosed herein comprise a mastoparan peptide and an adjuvant or carrier, or comprise modified mastoparan peptides.

As used herein, the terms “pest” and “pathogen” are used to identify organisms causative of disease. A “pest” generally relates to a macroscopic organism such as an insect or a mite. A “pathogen” generally relates to disease-causing microorganisms such as fungi, bacteria, and viruses.

As used herein, the term “effective amount” refers to an amount of an active ingredient, e.g., a bioactive composition comprising a modified mastoparan peptide, with or without an adjuvant, as appropriate under the circumstances, or comprising a mastoparan peptide and an adjuvant or carrier, wherein such amount as is capable of performing the function of the compound or property for which an effective amount is expressed. As will be pointed out below, the exact amount required will vary from process to process, depending on recognized variables such as the compounds employed, and the processing conditions observed. Thus, it is not possible to specify an exact “effective amount.” However, an appropriate effective amount may be determined by one of ordinary skill in the art using only routine experimentation. An effective amount can be administered in one or more administrations, applications, or dosages. A therapeutically effective amount of a bioactive composition described herein may be readily determined by one of ordinary skill in the art, and provides a measurable benefit to a plant or plant part, such as protecting the plant or plant part from subsequent challenge with a pathogen.

The term “treating,” as used herein, refers to ameliorating, improving, or remedying a disease, disorder, condition or symptom of a disease, disorder, or condition.

The term “preventing” means stopping or hindering a disease, disorder, condition, or symptom of a disease, disorder, or condition.

The bioactive compositions disclosed herein may be administered to a plant or plant part by direct injection, genetically through the use of transgenic plants, viral vectors, synthetic microbes, modified plant cells expressing a mastoparan peptide together with plant growth regulator genes to initiate autonomous cell division referred to as a symbiont; or by other means known to those skilled in the art when the mastoparan peptide is encoded in a plasmid, vector, or virus as a recombinant nucleic acid DNA or RNA, either alone or in combination with other peptides, signal peptides or as fusion with other biomolecules.

As used herein, the term “plant or plant part” includes reference to whole plants, plant organs (e.g., leaves, stems, roots, etc.), seeds, plant cells, plant germplasms, and progeny of the same. The term “plant cell” includes, without limitation, seeds suspension cultures, embryos, meristematic regions, callus tissue, leaves, roots, shoots, gametophytes, sporophytes, pollen, and microspores.

The term “Agrobacterium” as used herein refers to a soil-borne, Gram-negative, rod-shaped phytopathogenic bacterium. The cells are normally rod-shaped (0.6-1.0 m by 1.5-3.0 m), occur singly or in pairs, without endospore, and are motile by one to six peritrichous flagelia. Considerable extracellular polysaccharide slime is usually produced during growth on carbohydrate-containing media.

Agrobacterium-mediated transformation is a process of using Agrobacterium tumefaciens to transfer a gene of interest into host cells, generally plant but not limited to that kingdom.

Hereafter, plants will be discussed as the host organism. In a transient transformation the transferred DNA remains transiently in the nucleus while still being transcribed into desirable gene products. In a stable transformation the transferred DNA is integrated into the plant genome for inheritance into the next generation, generating transgenic plants.

As used herein, the term “symbiont” refers to a plant cell or a plurality of plant cells comprising at least one polynucleotide encoding at least one phytohormone biosynthetic enzyme and a polynucleotide of interest. The at least one phytohormone biosynthetic enzyme may be a cytokinin biosynthetic enzyme and/or an auxin biosynthetic enzyme. The cells of a symbiont autonomously divide due to the expression of the at least one polynucleotide encoding at least one phytohormone biosynthetic enzyme. A symbiont may comprise any number of cells, from 1 cell to 100,000 or more cells. The cells of a symbiont autonomously divide, forming an undifferentiated multi-cellular structure on a plant. The undifferentiated multicellular structure (symbiont) that is formed may be visually similar to a burl, a plant food body, a dormatia, an extrafloral nectary, a nodule, a plant neoplasm, or a gall, but are biochemically/genetically distinct by at least the transgenes expressed in the symbiont.

A symbiont may be removed from the original host plant, cultured in a laboratory setting, and/or transplanted onto another plant. When the symbiont, or at least one cell from the symbiont, is cultured, the “child symbiont material” may be used to refer to the new symbiont material formed over time and propagated from the original material removed from the host plant.

The terms “coding sequence,” “coding region,” and “open reading frame” are used interchangeably herein and refer to a region of continuous sequential nucleic acid triplets encoding a protein, a polypeptide, or a peptide sequence.

The terms “polyadenylation signal” and “polyA signal” are used interchangeably herein and refer to a nucleic acid sequence located 3′ to a coding region that promotes the addition of adenylate nucleotides to the 3′ end of an mRNA transcribed from the coding region.

The terms “promoter” and “promoter region” are used interchangeably herein and refer to a nucleic acid sequence, usually found 5′ to a coding sequence, that alter expression of the coding sequence by providing a recognition site for RNA polymerase and/or other recognition sites for other transcription-related factors utilized to produce RNA and/or initiate transcription at the correct site on the DNA.

The terms “recombinant nucleic acid vector” and “vector” are used interchangeably herein and refer to any agent such as a plasmid, cosmid, virus, autonomously replicating sequence, phage, or linear or circular single- or double-stranded DNA or RNA nucleotide segment, derived from any source, capable of genomic integration or autonomous replication, comprising a nucleic acid molecule in which one or more nucleic acid sequences have been linked in a functionally operative manner. Such recombinant nucleic acid vectors or constructs typically comprise a 5′ regulatory sequence or promoter region and a coding sequence encoding for a desired gene product. The vectors are typically designed such that once delivered into a cell or tissue, the coding sequence is transcribed into mRNA, which is optionally translated into a polypeptide or protein.

As used herein, the term “regeneration” refers to the process of growing a plant from a plant cell or tissue.

As used herein, the term “endogenous” refers to materials originating from within the organism or cell.

As used herein, the term “exogenous” refers to materials originating from outside of the organism or cell. As used herein, exogenous is intended to refer to any nucleic acid from a source other than the recipient cell or tissue, regardless of whether a similar (but not identical) nucleic acid may already be present in the recipient cell or tissue.

As used herein, the term “phenotype” refers to a trait exhibited by an organism resulting from the expression (or lack of expression) of nucleic acids in the genome (including non-genomic DNA and RNA such as plasmids and artificial chromosomes) and/or organelles of the organism.

As used herein, the term “transgenic” refers to organisms that have been stably transformed with an exogenous nucleic acid.

As used herein, the term “plant part” refers to any part of a plant including but not limited to the shoot, root, stem, seeds, stipules, leaves, petals, flowers, ovules, bracts, branches, petioles, internodes, bark, pubescence, tillers, rhizomes, fronds, blades, pollen, stamen, and the like. The two main parts of plants grown in some sort of media, such as soil, are often referred to as the “above-ground” part, also often referred to as the “shoots”, and the “below-ground” part, also often referred to as the “roots”. “Freshly emerging shoots” are shoots that have appeared as new growth on a plant in about the last 1 day, 2 days, 3 days, 4 days, 5 days, 6 days, 1 week, 2 weeks, 3 weeks, or 4 weeks.

Mention of trade names or commercial products in this disclosure is solely for the purpose of providing specific information and does not imply recommendation or endorsement by the U.S. Department of Agriculture.

Toxins represent a source of potential antimicrobials. They are contained in venoms and have evolved (e.g., in plants, animals and microbes) as part of defensive strategies. Typically, crude venoms contain a wide range of different peptide toxins, many of which are small in size, easy to synthesize, structurally stable, and highly biodiverse (RJ Lewis and ML Garcia, 2003, “Therapeutic potential of venom peptides,” Nat. Rev. Drug Discov. 2(10): 790-802). Antimicrobial peptides (AMPs), a promising class of antibiotic candidates, have been found in venoms (MDT Torres et al., 2018, “Structure-function-guided exploration of the antimicrobial peptide polybia-CP identifies activity determinants and generates synthetic therapeutic candidates,” Commun. Biol. 1: 221). AMPs have an almost unlimited sequence space and multiple mechanisms of action against microorganisms. These agents can present broad- or narrow-spectrum activity, and rapid antimicrobial and immunomodulatory properties, which allow them to act against a range of microorganisms, including Gram-negative and Gram-positive bacteria, fungi, viruses, and parasites. Relevant to their potential clinical translatability, bacteria exposed to AMPs have been shown to develop resistance at a much lower rate than when exposed to conventional antibiotics.

Mastoparans are a group of small wasp linear cationic-helical peptides. They are found in the venom of several species of social and solitary wasps originally described for promoting deregulation and release of histamine in mast cells. Mastoparans show a variety of biological effects including mast cell degranulation, activation of protein G and/or phospholipases A2, C, and D, serotonin and insulin release, and antimicrobial, hemolytic, and anticancer activities. For a review, see Correia de Santana C J et al. (2022, “Mastoparans: A Group of Multifunctional-helical Peptides with Promising Therapeutic Properties,” Front. Mol. Biosci. 9: 824989). But the effect of mastoparan peptides on plant pests has yet to be determined.

The disclosure provides bioactive compositions comprising at least one mastoparan peptide and a carrier and/or adjuvant, or comprising at least one modified mastoparan peptide.

The bioactive compositions taught herein may be used in methods to reduce or ameliorate infection in a subject in need thereof. Methods taught herein may reduce damage of plants caused by Liberibacter infection. In some embodiments, the bioactive compositions comprising at least one mastoparan peptide and a carrier and/or adjuvant, or comprising at least one modified mastoparan peptide may reduce or ameliorate Citrus Greening Disease in a Citrus plant in need thereof, or may reduce or ameliorate Zebra Chip Disease in a Solanaceae plant in need thereof.

Citrus greening, also known as huanglongbing (HLB; yellow dragon disease or yellow shoot disease) is caused by a phloem-limited bacterial infection (Candidatus liberibacter asiatcus, Candidatus liberibacter africanus, and Candidatus liberibacter americanus) spread by their insect vectors, which include Diaphorina citri and Trioza erytreae. The bacterium is introduced by psyllid feeding on new flush and spreads systematically through the plant vasculature, spreading throughout the canopy, stem, and root system in a matter of months. Over several years, the bacterium degrades the root and vascular system of the tree leading to blotchy mottling of leaves, reduced fruit production, early fruit drop of bitter, undeveloped fruit, and eventual plant death, typically in about 5 to 10 years.

Zebra chip is caused by a bacterial infection (Candidatus Liberibacter solanacearum) and vectored by at least five psyllid species (Bactericera cockerelli, Aphalara loca, Aphalara persicaria, Aphalara curta, Heterotrioza chenopodii) the disease from plant to plant. Zebra chip damages plants and produce of many Solanaceae crops including potato, tomato, carrot, and other vegetables.

The mastoparan peptides described herein may be modified by at least one of an N-terminal modification, an N-terminal substitution, a C-terminal modification, a C-terminal substitution, a presence of a D-amino acid, a presence of an unnatural amino acid, a cyclization, a backbone modification, a nanoparticle formulation. The mastoparan peptides described herein may have a plurality of these modifications.

The mastoparan peptides described herein may be delivered to plants through the use of transgenic plants, viral vectors, synthetic microbes, modified plant cells expressing a gene or genes of interest together with plant growth regulator genes to initiate autonomous cell division, or by any means known to those skilled in the art, when the mastoparan peptide is encoded in a plasmid, vector, or virus as a recombinant nucleic acid DNA or RNA, either alone or in combination with at least one other peptide or signal peptide, or as fusion with other biomolecules.

Any methodology known in the art to insert or otherwise modify the cellular DNA to introduce a mastoparan peptide to a plant or plant part can be used. For example, a disarmed Ti plasmid, containing a genetic construct for insertion of a mastoparan peptide, in Agrobacterium tumefaciens can be used to transform a plant cell, and thereafter, a transformed plant can be regenerated from the transformed plant cell using procedures well known in the art. See for example, EP 0116718, EP 0270822, PCT publication WO 84/02913, EP 0242246, U.S. Pat. Nos. 8,334,139, 5,352,605, and 6,174,724. Other types of vectors can be used to transform a plant cell, using procedures such as symbiont technology described, for example, in WO 2021055656; direct gene transfer, described, for example in US 20230399603, US 20220304273, and EP 0233247; pollen mediated transformation, described, for example in EP 0270356, PCT publication WO 1985001856, and U.S. Pat. No. 4,684,611; plant RNA virus-mediated transformation, described, for example in EP 0067553 and U.S. Pat. No. 4,407,956; liposome-mediated transformation, described, for example in U.S. Pat. No. 4,536,475; and other transformation methods such as those described in U.S. Pat. No. 6,140,553; M Fromm et al., 1990, “Inheritance and Expression of Chimeric Genes in the Progeny of Transgenic Maize Plants,” Nat. Biotechnol. 8: 833-839; WJ Gordon-Kamm et al., 1990, “Transformation of Maize Cells and Regeneration of Fertile Transgenic Plants,” Plant Cell 2(7): 603-618; K Shimamoto et al., 1989, “Fertile transgenic rice plants regenerated from transformed protoplasts,” Nature 338: 274-276; SK Datta et al., 1990, “Genetically engineered fertile indica-rice recovered from protoplasts,” Nat. Biotechnol. 8: 736-740; PCT publication WO 1992009696; PCT publication WO 2000071733; or US 20220002746.

Transgenic plants expressing the mastoparan peptides taught herein can be used in a conventional plant breeding scheme to produce more transgenic plants with the same characteristics, or to introduce genetic alteration(s) in other varieties of the same or related plant species. Seeds, which are obtained from the transformed plants, can contain the genetic alteration(s) as a stable sequence encoding a mastoparan inserted in chromosomal or organelle DNA. Plants comprising the genetic alteration(s) in accordance with the disclosure include plants comprising or derived from, root stocks of plants comprising the genetic alteration (staught herein. Hence, any non-transgenic grafted plant parts inserted on a transformed plant or plant part expressing a mastoparan peptide as taught herein are included in the disclosure.

A non-integrated expression system can be used to induce expression of one or more mastoparan peptides. Expression systems can include, for example, an origin of replication or autonomously replicating sequence (ARS) and expression control sequences, a promoter, an enhancer, and necessary processing information sites such as ribosome-binding sites, RNA splice sites, polyadenylation sites, transcriptional terminator sequences, and mRNA stabilizing sequences. Signal peptides from secreted polypeptides of the same or related species can also be included where appropriate, which will allow the mastoparan peptide to cross and/or lodge in cell membranes, cell walls, or be secreted from the cell.

Selectable markers useful in practicing the methodologies of this disclosure can be positive selectable markers, negative selectable markers, or screenable markers. Typically, positive selection refers to the survival of a genetically altered cell in the presence of a toxic substance only if the recombinant polynucleotide of interest is present within the cell. Positive selectable markers, negative selectable markers, and screenable markers are well known to those skilled in the art. One of skill in the art will recognize that any relevant marker available can be utilized in practicing the matters disclosed herein.

Screening and molecular analysis of recombinant organisms (e.g., transgenic plants or recombinant bacteria) comprising the mastoparan peptides taught herein can be performed utilizing any method known to one of skill in the art. For example, screening and molecular analysis of such recombinant organisms may be performed using nucleic acid hybridization techniques. The particular hybridization techniques are not essential to the subject disclosure. As improvements are made in hybridization techniques, they can be readily applied by one of skill in the art. Hybridization probes can be labeled with any appropriate label known to those of skill in the art. Hybridization conditions and washing conditions, for example temperature and salt concentration, can be altered to change the stringency of the detection threshold. Additionally, screening and molecular analysis of recombinant organisms, as well as creation of desired isolated nucleic acids can be performed using Polymerase Chain Reaction (PCR). PCR is a technique well-known and commonly used by those skilled in this art.

Although Citrus is susceptible to multiple different pathogens, Candidatus Liberibacter asiaticus (CLas) is currently the pathogen of greatest concern in the United States. CLas, an alpha-proteobacterium, has been associated with the disease known as Huanglongbing (HLB or citrus greening). The disease has been found to affect all members of the genus Citrus and is systemic in the plant. Therefore, symptoms can be found on the leaves, fruit, and roots. Symptoms include leaf mottling, yellow shoots, and the production of small lopsided fruit with a bitter taste and aborted seeds. Twig/branch dieback, increased early fruit drop, and death of the tree are all markers of advanced disease states. The disease is primarily spread as a result of transfer of the bacterium via feeding by Diaphorina citri Kuwayama (Asian citrus psyllid) although transmission via grafting and dodder (Cuscuta pentagona) is also possible.

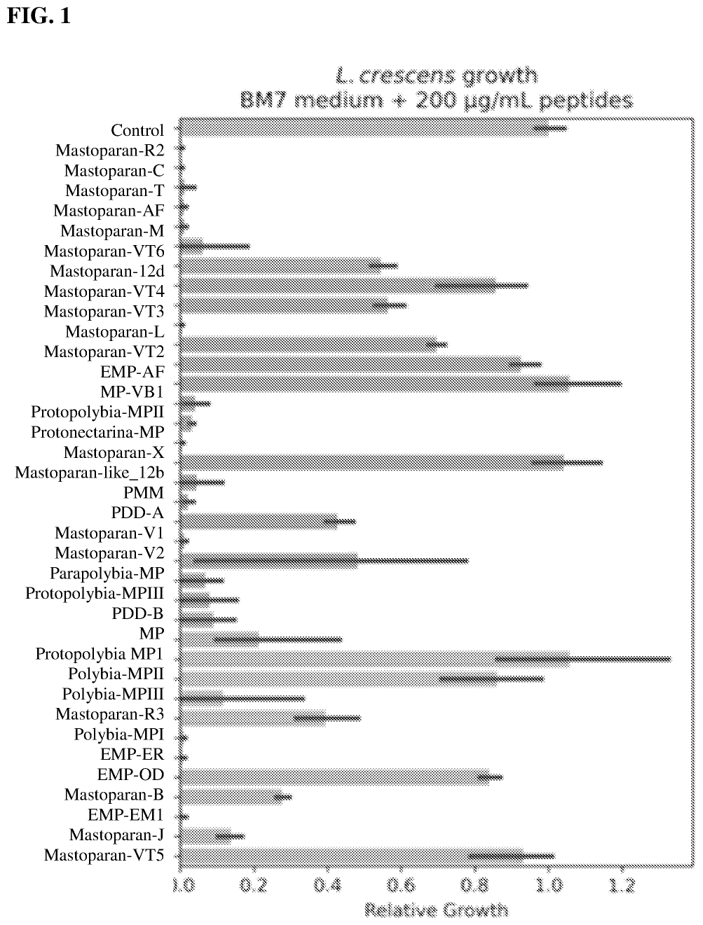

Forty one Mastoparan peptides synthesized with N-terminal acetylation and C-terminal amidation were resuspended in potassium phosphate buffer and tested for their effect on L. crescens BT-1 growth. The measured minimal inhibitory concentration (MIC) on L. crescens BT-1 was defined as the lowest tested concentration with optical density at 5 days less than 25% that of the control tested in the same experiment, and the results shown in Table 2. Peptides were added at 100, 200, 400, and 1000 μg/mL with 4, 3, 3, and 2 biological replicates each, respectively. FIG. 1 depicts a graph of the L. crescens growth in media treated with 200 g/mL of the compounds indicated to the left (control or mastoparan). The X axis shows the relative growth, and the Y axis shows the compounds tested.

As seen on Table 2, MICs ranged from about 100 to over 1,000 μg/mL. Diverse members of the Mastoparan family were relatively potent. In some cases small sequence changes greatly altered potency. For Example, Mastoparan-L which has the amino acid sequence INLKALAALAKKIL (set forth in SEQ ID NO: 10) presented with a MIC of 200 μg/mL, while Mastoparn-VT4 which has the amino acid sequence INLKAIAPLAKKLL (set forth in SEQ ID NO: 9), presented with a MIC of over 1,000 μg/mL. These results show that while Mastoparans have a high potential to treat plant diseases caused by L. crescens and closely-related bacteria, the specific Mastoparans that will provide effective control against any given pathogen requires further testing. The results also show that peptides with very similar amino acid sequences do not necessarily behave in the same manner.

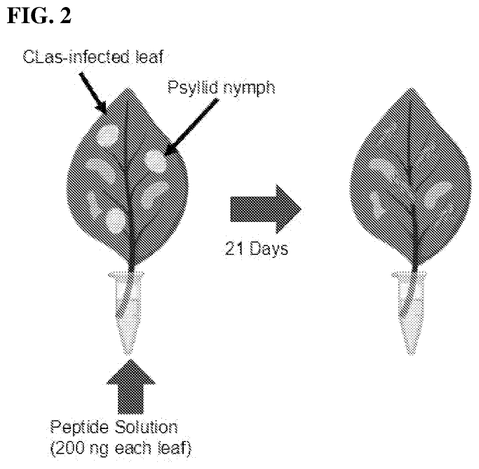

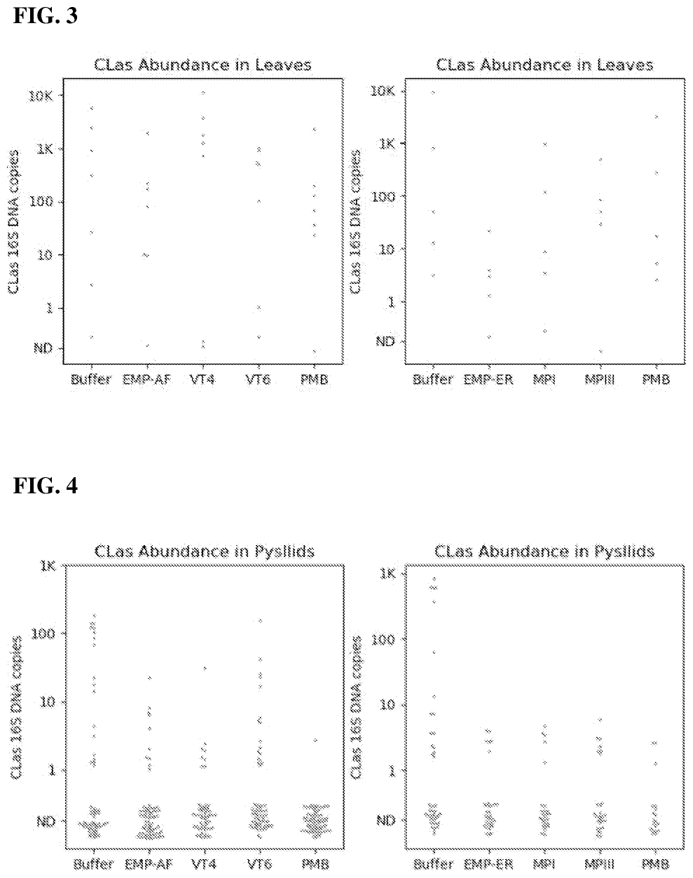

Several of the peptides tested in Example 1 were selected for follow-up experiments to support their potential role in prevention of CLas spread by D. citri. The peptides were tested in a detached leaf assay, a schematic representation of which is shown in FIG. 2. As seen in FIG. 3, leaf samples confirmed a similar level of CLas abundance for the leaves used in each treatment. Note also that for all treatments, leaves had widely divergent CLas abundances. A graph of the CLas abundances from the adult psyllids at the end of the experiment are presented in FIG. 4. This figure shows that the bacteria in mastoparan- and PMB-treated leaves were less able to infect the psyllid nymphs than bacteria in the buffer control-treated leaves. In two independent experiments approximately 17% of individual psyllids reared on leaves watered with the buffer control were highly infected with CLas (greater than 10 16S DNA copies per 50 ng of total nucleotides). Between 0% and 8% of psyllids reared on mastoparan-treated leaves reached this high level of infection. In both experiments, a Kruskal-Wallis test on CLas abundances confirmed a statistically significant effect across the experiment (pValues of 1.7e-17 and 0.06, respectively) and a Fisher Exact test on proportion of highly infected psyllids returned pValues less than 0.1 for mastoparan and positive control treatments except for mastoparan VT6 (pValue=0.11).

The results described in this disclosure show that treatment of a plant in need thereof with some modified mastaparan resulted in bacteria less able to infect the psyllid nymphs, and in preventing or ameliorating the spread of CLas by D. citri. Specifically, treatment with EMP-AF, mastoparan VT4, mastoparan VT6, EMP-ER, protopolybia MPI or polybia MPI, and protopolybia MPIII or polybia MPIII resulted in prevention or amelioration of the spread of CLas by D. citri.

Thus, in view of the above, there is described (in part) the following:

A bioactive composition comprising at least one modified mastoparan peptide.

The bioactive composition described above, wherein the at least one modified mastoparan peptide is modified by an N-terminal modification, an N-terminal substitution, a C-terminal modification, a C-terminal substitution, a presence of a D-amino acid, a presence of an unnatural amino acid, cyclization, or a mixture thereof.

The bioactive composition described above, wherein the at least one mastoparan peptide is modified by an N-terminal modification, wherein the N-terminal modification is acetylation, methylation, myristylation, or palmitoylation.

The bioactive composition described above, wherein the at least one mastoparan peptide is modified by a C-terminal modification, wherein the C-terminal modification is addition of an acid, an amide, an ester, an aldehyde, a para-nitroanilide, 7-amino-4-methylcoumarin, hydrazide, hydroxamic acid, or chloromethyl ketone.

The bioactive composition described above, wherein the at least one modified mastoparan peptide has the amino acid sequence as set forth in any one of SEQ ID NO: 1 to SEQ ID NO: 41, or a mixture thereof.

The bioactive composition described above, wherein the at least one modified mastoparan peptide has the amino acid sequence as set forth in SEQ ID NO: 14; SEQ ID NO: 9; SEQ ID NO: 4; SEQ ID NO: 34; SEQ ID NO:31; SEQ ID NO: 18; or a mixture thereof.

The bioactive composition described above, wherein the at least one modified mastoparan peptide is expressed in a host cell.

The bioactive composition described above, wherein the host cell is a plant cell.

A plant biopesticide composition comprising at least one mastoparan peptide and an adjuvant or carrier.

The plant biopesticide composition described above, wherein the adjuvant or carrier is at least one of an extender, a solvent, a surfactant, a stabilizer, an anti-freezing agent, a preservative, an antioxidant, a viscosity modifier, a suspending agent, a light absorber, a corrosion inhibitor, a pH-modifying substance, a lubricant, a plasticizer, a complexing agent, a colorant, a thickener, a solid adherent, a filler, a wetting agent, a dispersing agent, an anticaking agent, a diluent, or a combination thereof.

The plant biopesticide composition described above, wherein the at least one mastoparan peptide has the amino acid sequence as set forth in any one of SEQ ID NO: 1 to SEQ ID NO: 41, or a mixture thereof.

The plant biopesticide composition described above, wherein the at least one mastoparan peptide has the amino acid sequence as set forth in SEQ ID NO: 9; SEQ ID NO: 4; SEQ ID NO: 14; or a mixture thereof.

The plant biopesticide composition described above, wherein the at least one mastoparan peptide is expressed in a host cell.

The plant biopesticide composition described above, wherein the host cell is a plant cell.

A method for treating and/or ameliorating a pest and/or pathogen in a subject in need thereof, the method comprising treating the subject with an effective amount of bioactive composition comprising at least one modified mastoparan peptide to treat or ameliorate the pest and/or pathogen in the subject.

The method described above, wherein the modified mastoparan peptide in the bioactive composition is modified by an N-terminal modification, an N-terminal substitution, a C-terminal modification, a C-terminal substitution, a presence of a D-amino acid, a presence of an unnatural amino acid, cyclization, or a mixture thereof.

The method described above, wherein the mastoparan peptide in the bioactive composition is modified by an N-terminal modification, wherein the N-terminal modification is acetylation, methylation, myristylation, or palmitoylation.

The method described above, wherein the mastoparan peptide in the bioactive composition is modified by a C-terminal modification, wherein the C-terminal modification is addition of an acid, an amide, an ester, an aldehyde, a para-nitroanilide, 7-amino-4-methylcoumarin, hydrazide, hydroxamic acid, or chloromethyl ketone.

The method described above, wherein the at least one mastoparan peptide has the amino acid sequence as set forth in any one of SEQ ID NO: 1 to SEQ ID NO: 41, or a mixture thereof.

The method described above, wherein the at least one mastoparan peptide has the amino acid sequence as set forth in SEQ ID NO: 14; SEQ ID NO: 9; SEQ ID NO: 4; SEQ ID NO: 34; SEQ ID NO:31; SEQ ID NO: 18; or a mixture thereof.

The method described above, wherein the subject is a plant.

The method described above, wherein the plant is infected with a Liberibacter-species.

The method described above, wherein the plant infected with a Liberibacter-species is Citrus plant or a Solanaceae plant.

A method for treating and/or ameliorating a pest and/or pathogen in a subject in need thereof, the method comprising treating the subject with an effective amount of a plant biopesticide composition comprising at least one mastoparan peptide and an adjuvant or carrier to treat or ameliorate the pest and/or pathogen in the subject.

The method described above, wherein the adjuvant or carrier is at least one of an extender, a solvent, a surfactant, a stabilizer, an anti-freezing agent, a preservative, an antioxidant, a viscosity modifier, a suspending agent, a light absorber, a corrosion inhibitor, a pH-modifying substance, a lubricant, a plasticizer, a complexing agent, a colorant, a thickener, a solid adherent, a filler, a wetting agent, a dispersing agent, an anticaking agent, a diluent, or a combination thereof.

The method described above, wherein the at least one mastoparan peptide has the amino acid sequence as set forth in any one of SEQ ID NO: 1 to SEQ ID NO: 41, or a mixture thereof.

The method described above, wherein the at least one mastoparan peptide has the amino acid sequence as set forth in SEQ ID NO: 14; SEQ ID NO: 9; SEQ ID NO: 4; SEQ ID NO: 34; SEQ ID NO:31; SEQ ID NO: 18; or a mixture thereof.

The method described above, wherein the subject is a plant.

The method described above, wherein the plant is infected with a Liberibacter-species.

The method described above, wherein the plant infected by a Liberibacter-species is a Citrus plant or a Solanaceae plant.

Embodiments of the present disclosure are shown and described herein. It will be obvious to those skilled in the art that such embodiments are provided by way of example only. Numerous variations, changes, and substitutions will occur to those skilled in the art without departing from the disclosure. Various alternatives to the embodiments of the disclosure described herein may be employed in practicing the disclosure. It is intended that the included claims define the scope of the disclosure and that methods and structures within the scope of these claims and their equivalents are covered thereby. All publications, patents, and patent applications mentioned in this specification are herein incorporated by reference to the same extent as if each individual publication, patent, or patent application was specifically and individually indicated to be incorporated by reference.

### EXAMPLES

Having now generally described this disclosure, the same will be better understood by reference to certain specific examples, which are included herein only to further illustrate the disclosure and are not intended to limit the scope of the disclosure as defined by the claims.

### Example 1

### Effect of Peptides on Bacterial Growth

The effect of mastoparan peptides on Liberbacter crescens was studied. The peptides were synthesized with N-terminal acetylation and C-terminal amidation.

Growth of Liberbacter crescens strain BT-1 on BM7 medium with 200 μg/mL synthetic mastoparans, as compared to control cultures with no peptides added. Peptides were synthesized by Biomatik (Cambridge, Ontario, CA) with N-terminal acetylation and C-terminal amidation.

Peptides were resuspended in 10 mM potassium phosphate buffer, pH 5.6 at 20 mg/mi (or for some peptides not fully soluble at 20 mg/ml, a 10 mg/ml stock solution was prepared). L. crescens BT-1 was precultured in BM7 medium at 28° C. to an optical density (600 nm) of approximately 0.5 and diluted to GD 0.01 in 200 μL cultures in 96-well culture plates. Stock solutions of mastoparans were added to 200 μg/mL final concentration in BM7 medium, then cultures grown 5 days at 28° C. with constant shaking (250 rpm). Three biological replicates were tested for each peptide at this concentration, with nine replicates for the control condition.

FIG. 1 depicts a graph of the L. crescens growth. The X axis shows the relative grown, and the Y axis shows the compounds tested. Table 2, below, shows the peptides, observed solubility, and measured minimal inhibitory concentration (MIC) on L. crescens BT-1. MIC was defined as the lowest tested concentration with optical density at 5 days less than 25% that of the control tested in the same experiment. These data compiled from three separate experiments with peptides added at 100, 200, 400, and 1000 μg/mL with 4, 3, 3, and 2 biological replicates each, respectively.

TABLE 2

| | | | Solubility | MIC |
|---|---|---|---|---|
| Name | Identifier | Sequence | (mg/mL) | (μg/mL) |
| Mastoparan-R2 | SEQ ID NO: 1 | LNFKALAALAKKIL | >20 | 200 |
| Mastoparan-C | SEQ ID NO: 2 | LNLKALLAVAKKIL | >20 | 200 |
| Mastoparan-VT7 | SEQ ID NO: 3 | INLKAIAALARNY | < 5 | >200 |
| Mastoparan-VT6 | SEQ ID NO: 4 | INLKAIAALVKKLL | >20 | 100 |
| Mastoparan-AF | SEQ ID NO: 5 | INLKAIAALAKKLF | >20 | 200 |
| Mastoparan-T | SEQ ID NO: 6 | INLKAIAAFAKKLL | >20 | 200 |
| Mastoparan-like_12d | SEQ ID NO: 7 | INLKAIAAMAKKLL | >20 | 400 |
| Mastoparan-VT2 | SEQ ID NO: 8 | NLKAIAALAKKLL | >20 | 400 |
| Mastoparan-VT4 | SEQ ID NO: 9 | INLKAIAPLAKKLL | >20 | >1000 |
| Mastoparan-L | SEQ ID NO: 10 | INLKALAALAKKIL | 10 | 200 |
| Mastoparan-M | SEQ ID NO: 11 | INLKAIAALAKKLL | 10 | 200 |
| Mastoparan-VT3 | SEQ ID NO: 12 | INLKAITALAKKLL | 10 | 400 |
| MP-VB1t | SEQ ID NO: 13 | INMKASAAVAKKLL | >20 | >1000 |
| EMP-AF | SEQ ID NO: 14 | INLLKIAKGIIKSL | >20 | 1000 |
| Mastoparan-V2 | SEQ ID NO: 15 | INWKKIKSLIKAAMS | >20 | 200 |
| PDD-A | SEQ ID NO: 16 | INWKKIFEKVKNLV | >20 | 200 |
| Protonectarina-MP | SEQ ID NO: 17 | INWKALLDAAKKVL | >20 | 200 |
| Protopolybia-MPII | SEQ ID NO: 18 | INWKAIIEAAKQAL | >20 | 200 |
| PMM | SEQ ID NO: 19 | INWKKIASIGKEVLKAL | >20 | 200 |
| Mastoparan-V1 | SEQ ID NO: 20 | INWKKIKSIIKAAMN | >20 | 400 |
| Mastoparan-X | SEQ ID NO: 21 | INWKGIAAMAKKLL | >20 | 1000 |
| Parapolybia-MP | SEQ ID NO: 22 | INWKKMAATALKMI | >20 | >1000 |
| Mastoparan-like_12b | SEQ ID NO: 23 | INWKGIAAMKKLL | >20 | >1000 |
| Protopolybia-MPIII | SEQ ID NO: 24 | INWLKLGKAVIDAL | >20 | 200 |
| Mastoparan-R1 | SEQ ID NO: 25 | INWLKLGKKILGAI | >20 | 200 |
| Polybia-MPIII | SEQ ID NO: 26 | INWLKLGKMVMDAL | >20 | 400 |
| Mastoparan-R3 | SEQ ID NO: 27 | INWLKLGKQILGAL | >20 | 400 |
| Polybia-MPII | SEQ ID NO: 28 | INWLKLGKMVIDAL | >20 | 400 |
| PDD-B | SEQ ID NO: 29 | INWLKLGKKILGAL | >20 | 1000 |
| MP | SEQ ID NO: 30 | INWLKLGKKMMSAL | >20 | 1000 |
| Protopolybia-MPI | SEQ ID NO: 31 | INWLKLGKKVSAIL | >20 | 1000 |
| Venom_protein_13b | SEQ ID NO: 32 | IDWLKLGKMVMDVL | <5 | 200 |
| Polybia-MPI | SEQ ID NO: 33 | IDWKKLLDAAKQIL | >20 | 200 |
| EMP-ER | SEQ ID NO: 34 | FDIMGLIKKVAGAL | 10 | 200 |
| EMP-EF | SEQ ID NO: 35 | FDVMGIIKKIAGAL | <5 | 200 |
| EMP-OD | SEQ ID NO: 36 | GRILSFIKGLAEHL | >20 | 400 |
| EMP-EM2 | SEQ ID NO: 37 | LKLLGIVKKVLGAI | <5 | 200 |
| EMP-EM1 | SEQ ID NO: 38 | LKLMGIVKKVLGAL | 10 | 200 |
| Mastoparan-B | SEQ ID NO: 39 | LKLKSIVSWAKKVL | >20 | 400 |
| Mastoparan-VT5 | SEQ ID NO: 40 | VIVKAIATLSKKLL | >20 | >1000 |
| Mastoparan-J | SEQ ID NO: 41 | VDWKKIGQHILSVL | >20 | 400 |

As seen on Table 2, above, MICs range from 100 to over 1,000 μg/mL. Diverse members of the Mastoparan family were relatively potent. In some cases small sequence changes greatly altered potency. For Example, Mastoparan-L which has the amino acid sequence INLKALAALAKKIL (set forth in SEQ ID NO: 10) presented with a MIC of 200 μg/mL, while Mastoparn-VT4 which has the amino acid sequence INLKAIAPLAKKLL (set forth in SEQ ID NO: 9), presented with a MIC of over 1,000 μg/mL.

The information provided in this Example shows that while Mastoparans have a high potential to treat plant diseases caused by L. crescens and closely-related bacteria. The specific Mastoparans that will provide effective control against any given pathogen requires further testing. Furthermore, peptides with very similar amino acid sequences do not necessarily behave the same.

### Example 2

### Treatment of Infected Leaves

Several of the peptides tested in Example 1 were selected for follow-up experiments to support a potential role for certain mastoparans in prevention of the spread of CLas by D. citri.

Detached Leaf Assay for CLas infection of nymphs. This assay tests a treatment's ability to move in the plant vascular tissue to the CLas as well as the treatment's ability to interrupt the CLas life cycle sufficiently to stop infection of psyllid nymphs and propagative growth in the psyllid. Inhibition could occur by simply killing CLas or by modifying mobility, biofilm formation, etc. Testing multiple insects (10) per leaf and multiple leaves per treatment (7) ensured robust sampling of highly heterogenous CLas infection between leaves and from one part of the leaf to another. A schematic representation of a detached leaf assay for CLas infection of nymphs is shown in FIG. 2.

For this experiment, CLas-infected citron leaves with visible HLB symptoms were collected from colonies of CLas infected D. citri reared on citron. The petioles of these cut leaves were soaked in a solution containing 200 ng of a mastoparan peptide synthesized with N-terminal acetylation and C-terminal amidation in potassium phosphate buffer, in buffer only negative control, or in buffer with 200 ng polymixin-B, a small molecule known to inhibit CLas growth. After 24 hours for peptide absorption, 15 D. citri nymphs were added to each leaf (7 leaves per treatment) and they were monitored for 21 days as the nymphs fed on the leaves, molted, and matured into adult psyllids. At the end of the experiment all psyllids were individually tested for CLas bacteria by DNA extraction and quantitative PCR (qPCR) for the CLas 16S gene. Similarly, samples of each leaf were tested for CLas abundance.

As seen in FIG. 3, leaf samples confirmed a similar level of CLas abundance for the leaves used in each treatment. Note also that for all treatments, leaves had widely divergent CLas abundances. Because DNA is a long-lived molecule, these data do not distinguish between live or dead bacteria, thus they indicate that leaves used in each treatment had similar distribution of CLas abundances at the start of the experiment, but don't provide any information on the state of the bacteria in the leaves at the end of the experiment. Some of the treatments may have killed the bacteria, but the DNA persists in leaves for long periods.

A graph of the CLas abundances from the adult psyllids at the end of the experiment are presented in FIG. 4. This figure shows that the bacteria in mastoparan and PMB treated leaves were less able to infect the psyllid nymphs than bacteria in the buffer control leaves. Either the bacteria were simply less viable or dormant in the treated leaves, less able to move from the plant phloem into the psyllid digestive tract, or they were less able to proliferate in the adult psyllids after acquisition by nymphs. Note that in this experiment few individual psyllids in the buffer control had CLas abundance greater than 50 16S DNA copies per 50 ng extracted nucleotides. In some experiments a greater number of these highly infectious, high CLas abundance psyllids were observed than were observed here. Thus, in the present experiments there was less of an opportunity to observe a strong effect on the rate of highly infectious psyllids. Follow-up experiments may yield results with even stronger statistical significance. Even so, only one such highly infected psyllids were observed across all the mastoparan treatments, as opposed to 8 in the buffer control alone. All treatments showed a statically significant difference in the distribution of CLas abundances versus the buffer only control by the Kruskal-Wallis test. FIG. 4 presents plotted values of the number of CLas 16S DNA copies detected per 50 ng of DNA extracted from single adult psyllids. For values 1-10 or no CLas DNA detected, points have been artificially spread to allow visualization of the sample distribution.

The results obtained in this Example show that treatment of a plant in need thereof with a modified mastaparan resulted in bacteria less able to infect the psyllid nymphs, and preventing or ameliorating the spread of CLas by D. citri.

We claim:

1. A bioactive composition comprising at least one modified mastoparan peptide.

2. The bioactive composition of claim 1, wherein the at least one modified mastoparan peptide is modified by an N-terminal modification, an N-terminal substitution, a C-terminal modification, a C-terminal substitution, a presence of a D-amino acid, a presence of an unnatural amino acid, cyclization, or a mixture thereof.

3. The bioactive composition of claim 2, wherein the at least one mastoparan peptide is modified by an N-terminal modification, wherein the N-terminal modification is acetylation, methylation, myristylation, or palmitoylation.

4. The bioactive composition of claim 2, wherein the at least one mastoparan peptide is modified by a C-terminal modification, wherein the C-terminal modification is addition of an acid, an amide, an ester, an aldehyde, a para-nitroanilide, 7-amino-4-methylcoumarin, hydrazide, hydroxamic acid, or chloromethyl ketone.

5. The bioactive composition of claim 1, wherein the at least one modified mastoparan peptide has the amino acid sequence as set forth in any one of SEQ ID NO: 1 to SEQ ID NO: 41, or a mixture thereof.

6. The bioactive composition of claim 5, wherein the at least one modified mastoparan peptide has the amino acid sequence as set forth in SEQ ID NO: 14; SEQ ID NO: 9; SEQ ID NO: 4; SEQ ID NO: 34; SEQ ID NO:31; SEQ ID NO: 18; or a mixture thereof.

7. The bioactive composition of claim 1, wherein the at least one modified mastoparan peptide is expressed in a host cell.

8. The bioactive composition of claim 7, wherein the host cell is a plant cell.

9. A method for treating and/or ameliorating a pest and/or pathogen in a subject in need thereof, the method comprising treating the subject with an effective amount of the bioactive composition of claim 1 to treat or ameliorate the pest and/or pathogen in the subject.

10. The method of claim 9, wherein the modified mastoparan peptide in the bioactive composition is modified by an N-terminal modification, an N-terminal substitution, a C-terminal modification, a C-terminal substitution, a presence of a D-amino acid, a presence of an unnatural amino acid, cyclization, or a mixture thereof.

11. The method of claim 10, wherein the mastoparan peptide in the bioactive composition is modified by an N-terminal modification, wherein the N-terminal modification is acetylation, methylation, myristylation, or palmitoylation.

12. The method of claim 10, wherein the mastoparan peptide in the bioactive composition is modified by a C-terminal modification, wherein the C-terminal modification is addition of an acid, an amide, an ester, an aldehyde, a para-nitroanilide, 7-amino-4-methylcoumarin, hydrazide, hydroxamic acid, or chloromethyl ketone.

13. The method of claim 9, wherein the at least one modified mastoparan peptide has the amino acid sequence as set forth in any one of SEQ ID NO: 1 to SEQ ID NO: 41, or a mixture thereof.

14. The method of claim 13, wherein the at least one mastoparan peptide has the amino acid sequence as set forth in SEQ ID NO: 14; SEQ ID NO: 9; SEQ ID NO: 4; SEQ ID NO: 34; SEQ ID NO:31; SEQ ID NO: 18; or a mixture thereof.

15. The method of claim 9, wherein the subject is a plant.

16. The method of claim 15, wherein the plant is infected with a Liberibacter-species.

17. The method of claim 16, wherein the plant infected with a Liberibacter-species is citrus plant or a Solanaceae plant.

18. A plant biopesticide composition comprising at least one mastoparan peptide and an adjuvant or carrier.

19. The plant biopesticide composition of claim 18, wherein the at least one mastoparan peptide has the amino acid sequence as set forth in any one of SEQ ID NO: 1 to SEQ ID NO: 41, or a mixture thereof.


SEQUENCE LISTING (41 sequences)

SEQ ID NO: 1 - protein, 14 aa, Synthetic construct
LNFKALAALAKKIL

SEQ ID NO: 2 - protein, 14 aa, Synthetic construct
LNLKALLAVAKKIL

SEQ ID NO: 3 - protein, 13 aa, Synthetic construct
INLKAIAALARNY

SEQ ID NO: 4 - protein, 14 aa, Synthetic construct
INLKAIAALVKKLL

SEQ ID NO: 5 - protein, 14 aa, Synthetic construct
INLKAIAALAKKLF

SEQ ID NO: 6 - protein, 14 aa, synthetic construct
INLKAIAAFAKKLL

SEQ ID NO: 7 - protein, 14 aa, synthetic construct
INLKAIAAMAKKLL

SEQ ID NO: 8 - protein, 13 aa, synthetic construct
NLKAIAALAKKLL

SEQ ID NO: 9 - protein, 14 aa, synthetic construct
INLKAIAPLAKKLL

SEQ ID NO: 10 - protein, 14 aa, synthetic construct
INLKALAALAKKIL

SEQ ID NO: 11 - protein, 14 aa, synthetic construct
INLKAIAALAKKLL

SEQ ID NO: 12 - protein, 14 aa, synthetic construct
INLKAITALAKKLL

SEQ ID NO: 13 - protein, 14 aa, synthetic construct
INMKASAAVAKKLL

SEQ ID NO: 14 - protein, 14 aa, synthetic construct
INLLKIAKGIIKSL

SEQ ID NO: 15 - protein, 15 aa, synthetic construct
INWKKIKSLIKAAMS

SEQ ID NO: 16 - protein, 14 aa, synthetic construct
INWKKIFEKVKNLV

SEQ ID NO: 17 - protein, 14 aa, synthetic construct
INWKALLDAAKKVL

SEQ ID NO: 18 - protein, 14 aa, synthetic construct
INWKAIIEAAKQAL

SEQ ID NO: 19 - protein, 17 aa, synthetic construct
INWKKIASIGKEVLKAL

SEQ ID NO: 20 - protein, 15 aa, synthetic construct
INWKKIKSIIKAAMN

SEQ ID NO: 21 - protein, 14 aa, synthetic construct
INWKGIAAMAKKLL

SEQ ID NO: 22 - protein, 14 aa, synthetic construct
INWKKMAATALKMI

SEQ ID NO: 23 - protein, 13 aa, synthetic construct
INWKGIAAMKKLL

SEQ ID NO: 24 - protein, 14 aa, synthetic construct
INWLKLGKAVIDAL

SEQ ID NO: 25 - protein, 14 aa, synthetic construct
INWLKLGKKILGAI

SEQ ID NO: 26 - protein, 14 aa, synthetic construct
INWLKLGKMVMDAL

SEQ ID NO: 27 - protein, 14 aa, synthetic construct
INWLKLGKQILGAL

SEQ ID NO: 28 - protein, 14 aa, synthetic construct
INWLKLGKMVIDAL

SEQ ID NO: 29 - protein, 14 aa, synthetic construct
INWLKLGKKILGAL

SEQ ID NO: 30 - protein, 14 aa, synthetic construct
INWLKLGKKMMSAL

SEQ ID NO: 31 - protein, 14 aa, synthetic construct
INWLKLGKKVSAIL

SEQ ID NO: 32 - protein, 14 aa, synthetic construct
IDWLKLGKMVMDVL

SEQ ID NO: 33 - protein, 14 aa, synthetic construct
IDWKKLLDAAKQIL

SEQ ID NO: 34 - protein, 14 aa, synthetic construct
FDIMGLIKKVAGAL

SEQ ID NO: 35 - protein, 14 aa, synthetic construct
FDVMGIIKKIAGAL

SEQ ID NO: 36 - protein, 14 aa, synthetic construct
GRILSFIKGLAEHL

SEQ ID NO: 37 - protein, 14 aa, synthetic construct
LKLLGIVKKVLGAI

SEQ ID NO: 38 - protein, 14 aa, synthetic construct
LKLMGIVKKVLGAL

SEQ ID NO: 39 - protein, 14 aa, synthetic construct
LKLKSIVSWAKKVL

SEQ ID NO: 40 - protein, 14 aa, synthetic construct
VIVKAIATLSKKLL

SEQ ID NO: 41 - protein, 14 aa, synthetic construct
VDWKKIGQHILSVL